(19) United States
(12) Patent Application Publication
(10) Pub. No.: US 2005/0234516 A1
(43) Pub. Date: Oct. 20, 2005

(54) Treatment kit, composite structure, electric exciter, and cosmetic treatment method

(76) Inventor: Jean-Louis H. Gueret, Paris (FR)

Correspondence Address: Thomas L. Irving;FINNEGAN, HENDERSON, FARABOW, GARRETT, & DUNNER, L.L.P., 901 New York Avenue, N.W., Washington, DC, 20001-4413, US

(21) Appl. No.: 11/057,844

(22) Filed: Feb. 15, 2005

Related U.S. Application Data
(60) Provisional application No. 60/551,789, filed on Mar. 11, 2004.

(30) Foreign Application Priority Data
Feb. 16, 2004 (FR) 04 50279

(51) Int. Cl. A61N 1/18

(52) U.S. Cl. 607/3

(57) ABSTRACT

A treatment kit may include an electric exciter including at least two electrodes and a composite structure not including any electrodes. The composite structure may include a first surface configured to be applied to a zone to be treated and a second surface opposite from the first surface. The composite structure may include at least one active agent configured to exert an action on the zone to be treated. The composite structure may be configured to encourage migration of the active agent toward the first surface rather than toward the second surface. The electric exciter and the composite structure may be separate from one another and the treatment kit may be configured such that the at least two electrodes can be selectively brought into contact with the composite structure.

This application claims the benefit of priority under 35 U.S.C. § 119(e) of U.S. provisional application No. 60/551,789, filed on Mar. 11, 2004, the disclosure of which is incorporated herein by reference.

The present invention relates to a treatment kit for treating the body (e.g., the face) via a composite structure that may contain at least one active agent. For example, the present invention relates to a treatment kit including a composite structure and an electric exciter, and a cosmetic treatment method for treating the body cosmetically.

International patent application WO 02/24274 discloses a device for transdermal delivery of substances including a pouch provided with electrodes, which can be connected to a power supply unit. The pouch may include an opening rendering it possible to insert a liquid for administration into the pouch.

U.S. patent application publication no. 2002/0198484 discloses a transdermal electrotransport drug delivery device including, in a receptacle, two electrodes and two reservoirs. The electrodes are permanently in contact with their respective reservoirs and thus the electrodes and reservoirs are not separate from one another. Due to the permanent contact of the electrodes with the receptacles, the electrodes are not able to be brought into contact with the reservoirs, and the electrodes are not selectively removable with respect to the reservoirs.

There may exist a need to improve treatment kits for treating the body to possibly enhance the action and/or penetration of an active agent into a zone being treated. Further, there may exist a need to permit electrodes to be displaced relative to a composite structure including an active agent, for example, in order to tailor treatment to a zone being treated.

The invention may seek to satisfy one or more of the above-mentioned needs. Although the present invention may obviate one or more of the above-mentioned needs, it should be understood that some aspects of the invention might not necessarily obviate them.

In the following description, certain aspects and embodiments will become evident. It should be understood that the invention, in its broadest sense, could be practiced without having one or more features of these aspects and embodiments. It should be understood that these aspects and embodiments are merely exemplary.

In one aspect, as embodied and broadly described herein, the invention includes a treatment kit. The treatment kit may include an electric exciter including at least two electrodes and a composite structure not including any electrodes. The composite structure may include a first surface configured to be applied to a zone to be treated and a second surface opposite from the first surface. The second surface may be configured to be contacted by the at least two electrodes. The composite structure may include at least one active agent configured to exert an action on the zone to be treated. The at least one active agent may be located between the first surface and the second surface of the composite structure. The composite structure may be configured to encourage migration of the active agent toward the first surface rather than toward the second surface. The electric exciter and the composite structure may be separate from one another and the treatment kit may be configured such that the at least two electrodes can be selectively brought into contact with the composite structure.

Exemplary features of some embodiments of the invention may be desirable for a number of reasons. For example, some embodiments may include a treatment kit in which the electrodes may be easily displaced with respect to the composite structure. This may render it possible for the treatment to be adapted, for example, to the sensitivity of the zone to be treated. The some embodiments may also enhance the action and/or penetration of the active agent into the zone being treated. For example, flow of current between the electrodes may enhance opening and dilation of the pores of the skin and may render it it easier for the active agent to act on and/or penetrate into the skin. Some embodiments may further serve to activate microcirculation of the blood, improve muscle tone, and/or improve the healing capacity of the skin.

According to another aspect, the electric exciter and the composite structure may be configured such that the at least two electrodes may come into contact with the outside surface of the composite structure while making no significant contact with the at least one active agent. By configuring the composite structure so that the electrodes have no significant contact with the at least one active agent when they are in contact with the outside surface of the composite structure, some embodiments may substantially prevent the electrodes from being excessively soiled, thereby rendering is possible to reduce the risk of deposits forming on the electrodes. Such deposits, among other things, could lead to a reduction in the electrical conductivity of the electrodes. Some embodiments may also provide the user with the option of varying the magnitude of the electric current during treatment, for example, by pressing the electrodes to a greater or lesser extent against the composite structure.

In another aspect, each of the at least two electrodes may be associated with a handle such that the at least two electrodes can be brought substantially simultaneously into contact with the second surface of the composite structure via manipulation of the handle.

According to yet another aspect, the kit may include a packaging, wherein the electric exciter and the composite structure are packaged together within the packaging.

In still a further aspect, a treatment kit may include a composite structure including an outside surface and at least one active agent for exerting an action on a zone to be treated. The composite structure may be configured to be made electrically conductive. The treatment kit may include an electric exciter including a handle and at least two electrodes secured to the handle such that the at least two electrodes are able to be brought substantially simultaneously into contact with the outside surface of the composite structure, so as to cause an electric current to pass into the composite structure.

In still another aspect, the composite structure may not include any electrodes.

According to a further aspect, the composite structure may include at least one of a patch, a mask, and a cloth.

In yet another aspect, the composite structure may include a hydrophilic outer layer configured to be contacted by the at least two electrodes. For example, the outer layer may be dampened at the start of treatment, which may permit current to flow. When dry, the outer layer may not be electrically conductive.

According to a further aspect, the composite structure may include at least one layer including a woven fabric. According to another aspect, the composite structure may include at least one layer including a non-woven fabric. In still another aspect, the composite structure may include two outermost layers, each of the two outermost layers including a non-woven fabric. In yet a further aspect, the composite structure may include at least one layer including at least one of a gelled matrix and a polymeric matrix. For example, the at least one of a gelled matrix and a polymeric matrix may be a reservoir layer containing the at least one active agent. The reservoir layer, for example, may be situated between the two outermost layers.

According to another aspect, the at least one active agent may be substantially prevented from migrating toward the face against which the electrodes are contacted, for example, so as to reduce the risk of soiling the electrodes.

In still another aspect, the composite structure may include a reservoir layer and two additional layers. The reservoir layer may contain the at least one active agent and may be located between the two additional layers (e.g., two outermost layers). At least one of the two additional layers may be substantially free from any active agent prior to use. In still a further aspect, the composite structure may include a reservoir layer, a hydrophilic layer, and a layer that is one of hydrophilic and hydrophobic. The reservoir layer may contain the active agent and may be located between the hydrophilic layer and the layer that is one of hydrophilic and hydrophobic. At least the outermost layer that may be configured to come into contact with the electrodes may be substantially free from any active agent.

According to another aspect, the composite structure may include a reservoir layer and two additional layers (e.g., outermost layers). The two additional layers may have at least one of differing thicknesses, differing porosities, and differing properties. The reservoir layer may contain the active agent and may be located between the two additional layers. The differing properties may be, for example, an affinity for water and/or an affinity for the active agent to be contained in the reservoir layer.

In still a further aspect, the composite structure may include a reservoir layer and at least one additional layer. The reservoir layer may contain the at least one active agent and the at least one additional layer may be bonded to the reservoir layer.

According to another aspect, the at least one additional layer may define the outside surface of the composite structure. The reservoir layer may define a thickness, and the at least one additional layer defining the outside surface of the composite structure may define a thickness greater than the thickness of the reservoir layer. In another aspect, the at least one additional layer may include a layer of lower porosity defining the outside surface of the composite structure.

In yet a further aspect, the electrodes may be brought into contact with, for example, a thicker, outermost layer. Alternatively, or in addition, the electrodes may be brought into contact with a layer having a lower porosity. For example, the porosity of the layer defining the outside surface of the composite structure may be selected so that it is difficult for the active agent to diffuse into the layer such that while the composite structure is being used, the electrodes may remain without any significant contact with the active agent.

In yet another aspect, the composite structure may be configured to permit visual identification of the surface that is intended to come into contact with the skin and/or the surface that is intended to come into contact with the electrodes. For example, the composite structure may include surfaces having colors that differ from one another.

In a further aspect, the composite structure may be substantially dry prior to use. The composite structure may be dampened in order to make it conductive (e.g., electrically conductive).

In another aspect, prior to use, the at least one active agent may be in at least one of a dehydrated state, a freeze-dried state, and a crystallized state. For example, the at least one active agent may be in a particulate form.

In still another aspect, the composite structure may include a layer loaded with liquid. For example, the liquid may include at least one of water, alcohol, and propylene glycol. For example, the layer loaded with liquid (e.g., the reservoir layer) may include the at least one active agent.

In yet a further aspect, the composite structure may be configured to be non-adhesive when dry and adhesive when dampened.

In still another aspect, the composite structure may include a layer (e.g., the reservoir layer) including adhesive.

According to another aspect, composite structure may include any electrically conductive element (e.g., an electrolyte).

According to still another aspect, the composite structure may include magnetic particles and/or electrically-conductive particles. For example, the composite structure may include metallic particles (e.g., copper particles). In a further aspect, the magnetic particles may be coated and/or the magnetic particles may be in the form of fibers. In another aspect, the composite structure may include a reservoir layer containing the at least one active agent and one of a woven fabric including fibers and a non-woven fabric including fibers. At least a portion of the magnetic particles may be at least one of located in the reservoir layer and located in or between the fibers.

According to a further aspect, the composite structure does not necessarily include magnetic particles and/or electrically-conductive particles. In one aspect, the composite structure may include ions configured to provide conduction (e.g., electrical conduction).

In yet another aspect, the electrically-conductive particles and/or the magnetic particles may be located in the reservoir layer and/or in another layer of the composite structure. The particles may be retained between the fibers of a woven and/or a non-woven fabric, and/or the particles may be dispersed in a matrix of the composite structure (e.g., in a gelled matrix and/or in a polymeric matrix).

In a further aspect, the composite structure may include a reservoir layer, a hydrophilic layer, and a layer that is one of hydrophilic and hydrophobic. The reservoir layer may contain the active agent and may be located between the hydrophilic layer and the layer that is one of hydrophilic and hydrophobic.

According to another aspect, the composite structure may include a reservoir layer containing the at least one active agent. The reservoir layer may include at least one of a super-absorbent compound; a polyacrylate; an acrylic polymer; a vinyl polymer; a polyurethane; a latex; and a pseudolatex. In one aspect, the composite structure may include glycerine.

In another aspect, the at least one active agent may be a cosmetic, dermatological, and/or other agent configured for administration to human beings.

In still a further aspect, the active agent may include at least one compound selected from metals; metal alloys; cobalt; barium; chromium; aluminum; silver; copper; titanium; bronze; manganese; metallic oxides; iron oxides; ferrites; rare earths; silicates; sulfates; barium sulfates; carbonates; calcium carbonates; non-ferrous compounds; sulfur; magnesium; calcium; boron; potassium; carbon; oligo-elements; sea salt; rock salt; clay; steatite; algae and plankton and extracts therefrom; roots; liquorice; ginger; oily waxes; proteins; hormones; collagens; alums; alum stone; glucose; vitamins; vitamin C; vitamin A; Vitamin F; vitamin B; vitamin E; plant extracts; glycerin; laponite; wetting agents; collagen; acids; salicylic acid; thio acid; caffeine; essential aromatic oils; coloring agents; anti-oxidants; free radical scavengers; moisture absorbers; moisturizers; depigmenting agents; liporegulators; anti-acne agents; antidandruff agents; anti-ageing agents; softeners; antiwrinkle agents; keratolitic agents; anti-inflammatory agents; fresheners; healing agents; vascular protectors; antibacterial agents; antifungal agents; antiperspirants; deodorants; skin conditioners; anesthetics; immunomodulators; and nourishing agents.

In still a further aspect, the at least two electrodes may be associated with the electric exciter such that the at least two electrodes may be displaced relative to the electric exciter. For example, the at least two electrodes may be configured to be displaced relative to the electric exciter via at least one of sliding and turning.

According to another aspect, at least one of the electrodes (e.g., two of the electrodes) may be configured to be re-oriented relative to the handle.

In yet another aspect, the at least two electrodes may be separated by a distance from one another. In one aspect, the distance may be adjustable. In another aspect, at least one of the electrodes may be configured to be removed from the electric exciter. For example, the kit may include a plurality of removable electrodes having various shapes (e.g., electrodes configured to have different contact areas with the composite structure so that the electrodes may be selected and fixed onto the electric exciter, for example, as a function of the treatment to be performed, of the selected composite structure, and/or of the zone of the skin being treated).

In another aspect, the electric exciter may have electrodes that are not configured to be removed from the electric exciter.

In still another aspect, the at least two electrodes may be configured to be prevented from moving relative to one another other via an electrical insulator. For example, the electrical insulator may include a non-conductive material selected from elastomer, thermoplastic resin, and silicone.

In yet a further aspect, the at least two electrodes may be substantially free of sharp edges. In one aspect, each of the at least two electrodes may include a rounded end (e.g., a substantially spherical end).

In still another aspect, the two electrodes may include two conductors each provided with a rounded head. For example, the two conductors may each include thin rods.

According to some aspects, the electric exciter may be configured to be powered by at least one battery (e.g., a rechargeable battery). According to another aspect, the electric exciter may include a box configured to house at least one battery (e.g., a rechargeable battery). In another aspect, the electric exciter may be configured to be connected to an electrical adaptor connected to an electric outlet.

In a further aspect, the electric exciter may define an ergonomic shape (e.g., the electric exciter may include a box defining an ergonomic shape). For example, the electric exciter may have a shape that may be easily manipulated using one hand. According to one aspect, the electric exciter may define a handle at least partially including a battery.

In a further aspect, the electric exciter may include a switch. In another aspect, the electric exciter may include a pilot lamp configured to light up, for example, when current is passing through the electrodes. In a further aspect, the pilot lamp may include a two-color light-emitting diode (LED).

In yet another aspect, the electric exciter may be configured such that current flows intermittently.

In still a further aspect, the at least two electrodes may each include a terminal, and the electric exciter may be configured such that voltage at the terminals of the at least two electrodes is substantially identical to voltage at terminals of an electricity source associated with the electric exciter during use. In another aspect, the electric exciter may include a controller configured to permit raising and/or lowering of the voltage delivered by the electrical energy source.

According to yet a further aspect, the electric exciter may be configured such that voltage at terminals of the at least two electrodes is less than about 30 volts (e.g., about 9 volts).

In another aspect, the composite structure may include a patch through which electric current can flow to the skin.

In still another aspect, the electric exciter may include a control member configured to adjust electrical power. For example, the electric exciter may include a potentiometer and/or a multi-position switch configured to adjust, for example, the voltage and/or the current (e.g., the maximum current).

In yet another aspect, the electric exciter may include a vibrator configured to cause the at least two electrodes to vibrate. For example, the vibrator may be configured such that vibrations are transmitted to the composite structure and/or to the region of the body on which the composite structure is applied. According to another aspect, the electric exciter may include a selector configured to allow a user to selectively activate the vibrator. In another aspect, the electric exciter may include a selector configured to allow a user to choose an operating mode in which the at least two electrodes vibrate without being electrically charged.

In a further aspect an electric exciter may include a battery (e.g., a rechargeable battery), two electrodes, a vibrator configured to cause the two electrodes to vibrate, and a selector configured to selectively control the vibrator.

According to yet another aspect, the two electrodes may include two conductors each provided with a rounded head. For example, the two conductors may include thin rods.

In one aspect, the vibrator may include an electric motor configured to rotate an unbalanced mass. In another aspect, the electric exciter may include a controller, for example, a potentiometer, rendering it possible to adjust the amplitude and/or the frequency of electrode vibration.

According to another aspect, electric exciter may be substantially waterproof.

In yet another aspect, the composite structure and/or the electric exciter may be provided either alone or in combination (e.g., assembled together) in a case. In some aspects, the case may be provided through any sales channels, for example, in a shop and/or via correspondence and/or via a beauty parlor and/or a hairdressing salon.

In still a further aspect, a treatment kit may include an electric exciter having at least two electrodes and a composite structure that does not have any electrodes. The composite structure may include a first surface configured to be applied to the zone to be treated, a second surface opposite from the first surface and against which the electrodes may be contacted, and an active agent configured to exert an action on the zone to be treated. The active agent may be included in the composite structure between the first and second surfaces. The composite structure may be configured to encourage migration of the active agent toward the first surface rather than toward the second surface during use. For example, the active agent may be included in a reservoir layer located between two additional layers, one of which may be relatively more permeable to the active agent than the other of the additional layers. This may render it possible to reduce the risk of the electrodes coming into contact with the active agent, which may reduce the risk of soiling the electrodes.

In yet another aspect, both electrodes may be secured to a handle and both electrodes may be brought substantially simultaneously into contact with the second surface of the composite structure by manipulating the handle.

According still a further aspect, an electric exciter may include a battery, at least two electrodes, and a head configured to be rigidly secured to the battery. The head may be associated with the at least two electrodes such that the at least two electrodes are prevented from moving relative to one another, and the head may be configured to allow the at least two electrodes to be brought into substantially simultaneous contact with a composite structure.

In yet another aspect, the electric exciter may include a box defining a housing for receiving the battery.

According to still a further aspect, an electric exciter may include a box including a plastic material and being configured to receive at least one battery (e.g., a rechargeable battery) having terminals. The electric exciter may include two electrodes associated with the box, each electrode including an external rounded head and a rod configured to contact a respective terminal of the battery.

In yet another aspect, a treatment kit may include a composite structure configured to be made electrically conductive and including at least one active agent for exerting an action on a zone to be treated. The kit may include an electric exciter including at least two electrodes secured to a handle enabling them to be brought into contact with an outside surface of the composite structure so as to cause an electric current to pass into the composite structure and/or so as to transmit vibration to the composite structure.

According to yet another aspect, each of the two electrodes may have a substantially rounded end, for example, a substantially spherical end, formed of metal and/or a conductive elastomer disposed at the end of a flexible rod (e.g., a rod having a small diameter (e.g., a diameter ranging from about 0.5 millimeter to about 3 millimeters (e.g., ranging from about 1 millimeter to about 2 millimeters))).

In still another aspect, the two electrodes may be configured to cause an electric current to pass into the composite structure and/or to cause vibration to be transmitted thereto.

According to a further aspect, the electrodes may be interchangeable so as to render it possible for the user to choose between a plurality of electrodes, for example, depending on the type of treatment desired. For example, some elements forming the electrodes may be electrically conductive, while other elements forming the electrodes may not be electrically conductive.

According to a further aspect, a cosmetic treatment method for cosmetically treating a zone of the skin may include applying a composite structure to the zone of the skin to be treated. The composite structure may include at least one active agent and may include no electrodes. The method may include contacting the composite structure with an electric exciter including at least two electrodes. The composite structure may be configured to pass electric current into the composite structure without significant contact occurring between the at least two electrodes and the at least one active agent.

As used herein, the term “without significant contact” means that when the electrodes are removed from the composite structure, there is no significant amount of active agent remaining or the electrodes.

In a further aspect, the method may include dampening the composite structure before applying the composite structure to the zone to be treated. In still another aspect, the method may include dampening the skin prior to applying the composite structure to the zone of the skin to be treated.

According to yet another aspect, the composite structure may be configured to become adhesive upon contact with the skin. For example, the method may include dampening the skin before applying the composite structure in a dry state to the zone of the skin to be treated.

In still another aspect, during treatment, the at least two electrodes may be prevented from moving relative to the composite structure. In yet another aspect, during treatment, the at least two electrodes may be moved relative to the composite structure.

According to yet another aspect, the at least two electrodes may be in contact with the composite structure continuously during treatment. In still another aspect, the at least two electrodes may be in contact with the composite structure intermittently during treatment.

In one aspect, the at least two electrodes may be in contact with the composite structure for a period of time ranging from about 3 minutes to about 15 minutes.

In still another aspect, during treatment, the at least two electrodes may be caused to vibrate.

According to a further aspect, the composite structure may be configured to be discarded after use.

In yet another aspect, a cosmetic treatment method for cosmetically treating a zone of the skin may include dampening a composite structure that does not have electrodes and that comprises at least one active agent, and bringing at least one electrode into contact with the composite structure so as to cause an electric current to pass through the composite structure.

According to some aspects, depending on the composite structure used (i.e., on the active agent(s) that the composite structure includes, on the applied current, and/or on the method of applying the electrodes against the composite structure), some embodiments of the invention may seek, for example, to eliminate pain, to treat a nervous condition, to treat eczema and/or herpes, to exert an action against wrinkles, bags, dryness, redness, acne, and/or age spots.

Still a further aspect may relate to a method of promoting the sale of at least one composite structure to be applied to the skin, which may include, for example, mentioning the possibility of using the composite structure in association with an electric current and/or electric exciter. Such promotion may be performed using any communication channel: For example, such promotion may be performed via a sales person directly at a point-of-sale, via radio, via television, and/or via telephone, for example, in the form of commercials and/or short messages (SMS). Such promotion may be performed via the written press (i.e., the news) and/or any other document for advertising purposes. In some examples, promotion may be performed via the Internet and/or via any other suitable computer network and/or mobile telephone network. Promotion may also occur directly on packaging (e.g., packaging for the kit or any component of the kit) and/or on any instructions associated therewith.

Aside from the structural and procedural arrangements set forth above, the invention could include a number of other arrangements, such as those explained hereinafter. It is to be understood, that both the foregoing description and the following description are exemplary.

The accompanying drawings are incorporated in and constitute a part of this specification. The drawings illustrate exemplary embodiments of the invention and, together with the description, serve to explain some principles of the invention. In the drawings,

FIG. 1 is a schematic side view of an exemplary use of an exemplary embodiment of a treatment kit;

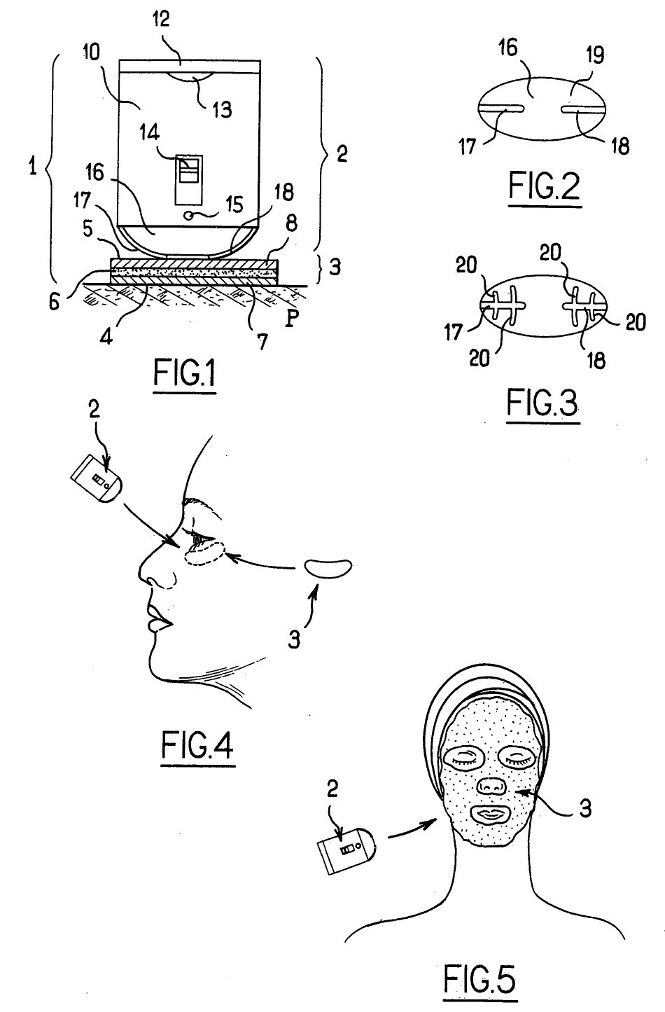

FIG. 2 is a schematic view of one end of an exemplary embodiment of an electric exciter;

FIG. 3 is a schematic view of one end of another embodiment of an electric exciter;

FIG. 4 is a schematic view of a further exemplary use of a further embodiment of a treatment kit;

FIG. 5 is a schematic view of another exemplary use of another embodiment of a treatment kit;

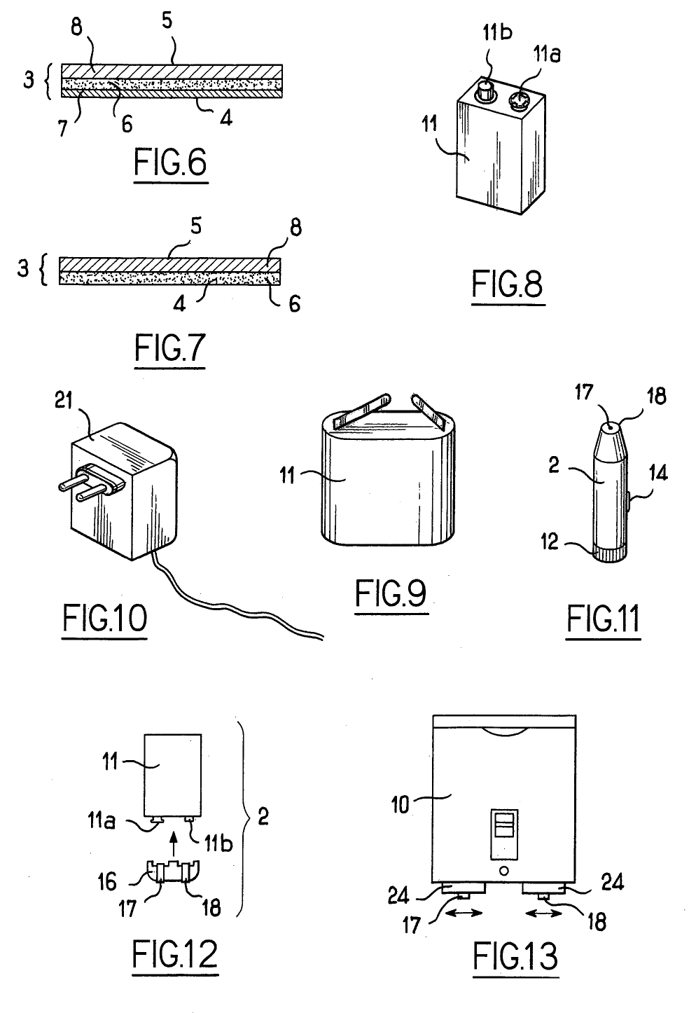

FIG. 6 is a schematic section view of an exemplary embodiment of a composite structure;

FIG. 7 is a schematic section view of a further embodiment of a composite structure;

FIG. 8 is a schematic perspective view of an exemplary embodiment of an electric energy source;

FIG. 9 is a schematic perspective view of a further embodiment of an electric energy source;

FIG. 10 is a schematic perspective view of another embodiment of an electric energy source;

FIG. 11 is a schematic perspective view of another embodiment of an electric exciter;

FIG. 12 is a schematic side view of another embodiment of an electric exciter;

FIG. 13 is a schematic side view of a further embodiment of an electric exciter;

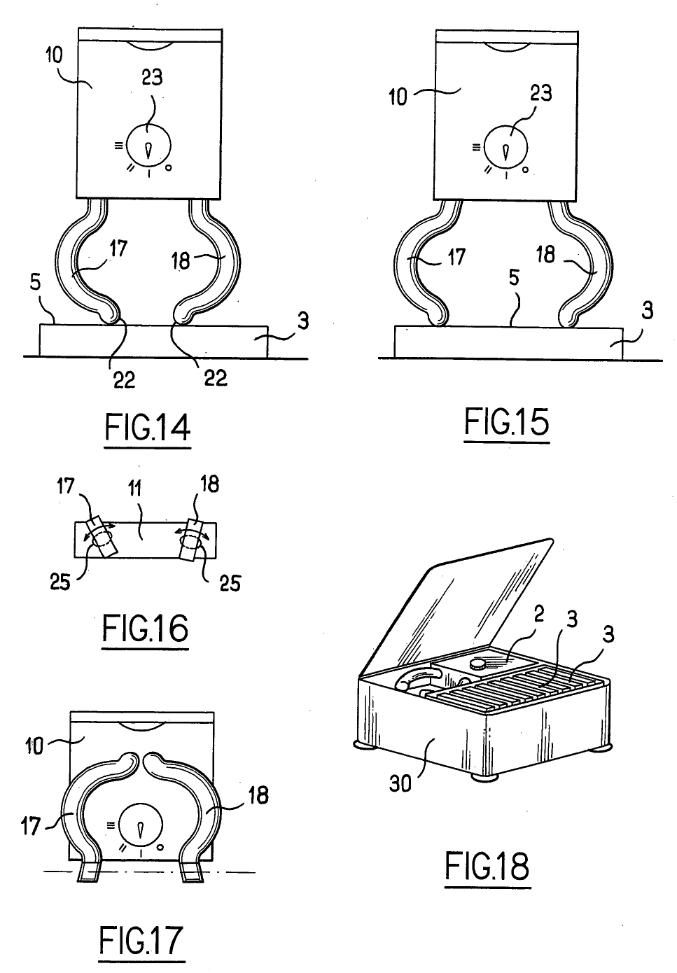

FIG. 14 is a schematic side view of another embodiment of a treatment kit including a further embodiment of an electric exciter in a first configuration;

FIG. 15 is a schematic side view of the treatment kit embodiment of FIG. 14 with the electric exciter in a second configuration;

FIG. 16 is a schematic end view of another embodiment of an electric exciter;

FIG. 17 is a schematic side view showing a further embodiment of an electric exciter;

FIG. 18 is a schematic perspective view of another embodiment of a treatment kit;

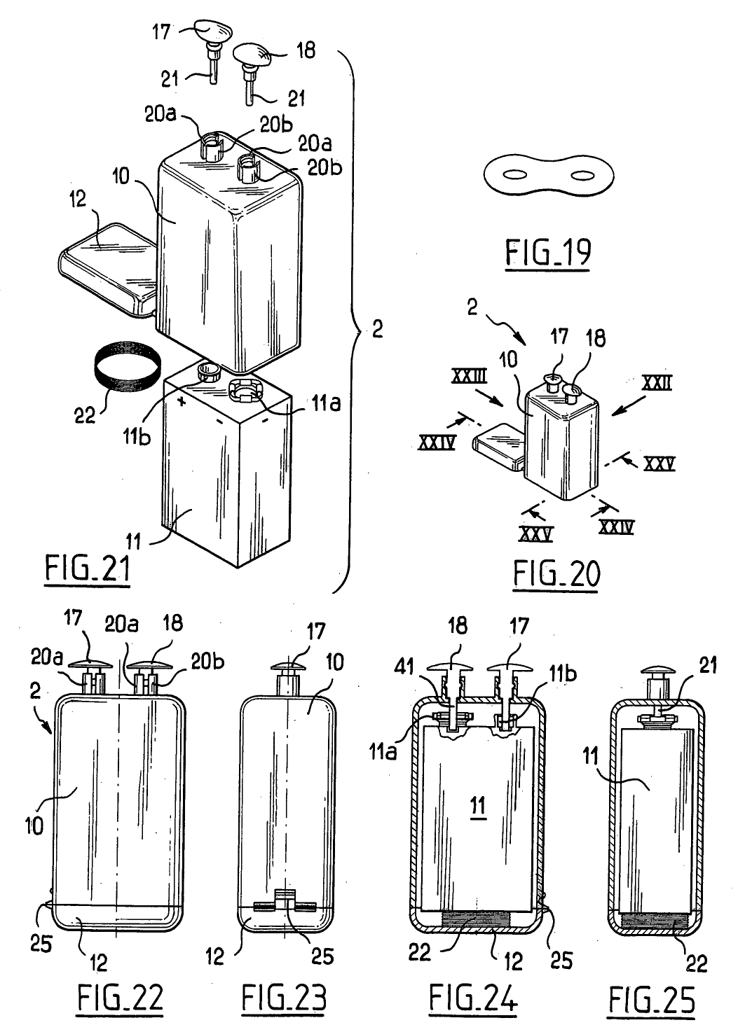

FIG. 19 is a schematic view of a further embodiment of a composite structure;

FIG. 20 is a schematic perspective view of a further embodiment of an electric exciter;

FIG. 21 is a schematic, exploded perspective view of the electric exciter embodiment of FIG. 20;

FIG. 22 is a view along line XXII of FIG. 20;

FIG. 23 is a view along line XXIII of FIG. 20;

FIG. 24 is a schematic, partial section view along line XXIV-XXIV of FIG. 20;

FIG. 25 is a schematic, partial section view along line XXV-XXV of FIG. 20;

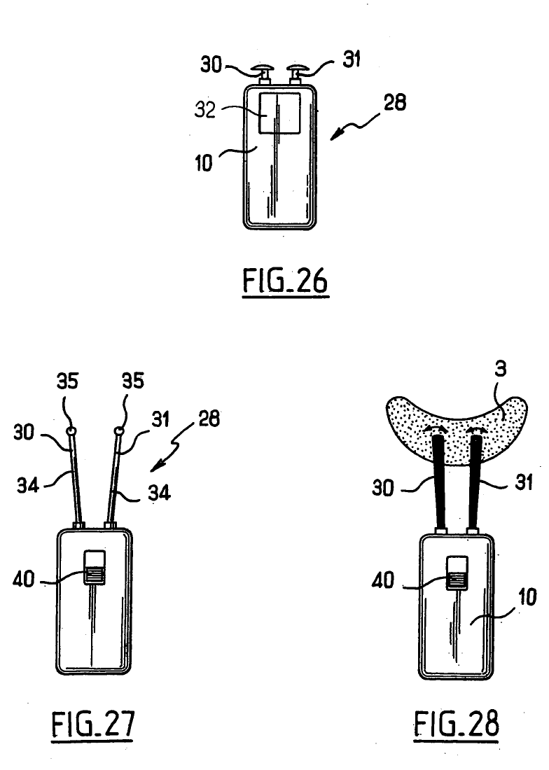

FIG. 26 is a schematic side view of a further embodiment of an electric exciter;

FIG. 27 is a schematic side view of another embodiment of an electric exciter in a first condition; and

FIG. 28 is a schematic side view of the electric exciter embodiment of FIG. 27 in a second condition.

Reference will now be made in detail to some possible embodiments of the invention, examples of which are illustrated in the accompanying drawings. Wherever possible, the same reference numbers are used in the drawings and the description to refer to the same or like parts.

FIG. 1 depicts an exemplary embodiment of an assembly 1 that may include an electric exciter 2 and a composite structure 3 configured be applied via a first surface 4 to a zone of skin P to be treated. The composite structure 3 may include a second surface 5 opposite from the first surface 4. The electric exciter 2 may be brought into contact with the second surface 5. For example, the composite structure 3 may be in the form of a patch, for example, as depicted in FIG. 4, a face mask, for example, as depicted in FIG. 5, and/or a mask for the eyes, for example, as depicted in FIG. 19. The composite structure 3 may be configured in still other forms, such as, for example, forms having a contour adapted to the shape of the zone to be treated and/or forms such as a disk, a strip, and/or a cloth, although other configurations are contemplated.

According to some embodiments, the composite structure 3 may include at least one active agent for exerting an action on the skin and/or for some other action. The at least one active agent may be suitable for being contained (e.g., as shown in FIG. 1) in a reservoir layer 6 situated between a first layer 7 and a second layer 8, which may define the first and second surfaces 4 and 5, respectively. For example, the reservoir layer 6 may include an adhesive matrix substantially ensuring cohesion between the first and second layers 7 and 8. In the exemplary embodiment shown in FIG. 1, the properties of the first and second layers 7 and 8 may be selected so as to encourage the at least one active agent to migrate toward the first surface 4, for example, rather than toward the second surface 5 when the composite structure 3 is in use. In order to achieve the selective transfer of the active agent toward the first surface 4, the first layer 7 may include large pores, for example, and the second layer 8 may be chosen to be less porous than the first layer 7.

As shown in FIG. 6, the composite structure 3 may define first and second layers 7 and 8 having differing thicknesses. For example, the second layer 8 may be thicker than the first layer 7, and the at least one active agent may be transferred toward the first surface 4 rather than toward the second surface 5. The second layer 8 may, for example, be about at least twice as thick as the first layer 7. According to some embodiments, the first layer 7 may be omitted altogether, for example, as shown in FIG. 7, and the reservoir layer 6 containing the active agent may define the first surface 4. In such embodiments, the second layer 8 may cover the reservoir layer 6 and may define the second surface 5.

The outermost layers 7 and 8 and the reservoir layer 6 may be of various types. For example, at least one of the layers may include a woven fabric, a non-woven fabric, a gelled matrix, a polymeric matrix, and/or still another media (e.g., a flexible media). Where desired, at least one of the layers may be at least partially metallized (e.g., aluminized). For example, at least one of the layers may include an aluminized non-woven fabric.

According to some embodiments, the reservoir layer 6 may include an adhesive based on a substance selected from vinyl, polyvinyl acrylate (PVA), polyvinyl pyrrolidone (PVP), pseudolatex, acrylic polymer, polyurethane, and/or latex elastomer. The reservoir layer 6 may include moisture absorbers, for example, polyacrylates and/or other super-absorbent compounds that may be capable of absorbing several times their own weight in liquid. For example, the reservoir layer 6 may include at least one material selected from acrylic polymer; vinyl polymer; polyurethane; latex; and pseudolatex.

In some embodiments, the composite structure is made in accordance with U.S. patent application publication No. U.S. 2001/0028894, for example, the content of which is incorporated herein by reference.

According to some embodiments, the composite structure 3 may include electrically-conductive particles, for example, particles of a metal such as, for example, copper, which may improve, for example, the conduction of electric current between electrodes 17 and 18, such as those shown in FIG. 1. This may improve the quality of the treatment. For example, metal particles may be dispersed in the reservoir layer 6 and/or elsewhere, and the metal particles may be retained between fibers of a woven fabric and/or a non-woven fabric.

In some embodiments, the composite structure 3 may include coated and/or uncoated magnetic particles, for example, so as to create a magnetic field. During use, the effects resulting from the magnetic particles may be combined with the effects of passing the electric current. For example, the active agent may include (or may be constituted by), at least one compound selected from metals; metal alloys; cobalt; barium; chromium; aluminum; silver; copper; titanium; bronze; manganese; metallic oxides; iron oxides, ferrite; rare earths; silicates; sulfates; barium sulfate; carbonates; calcium carbonates; non-ferrous compounds; non-ferrous compounds of sulfur, magnesium, calcium, boron, potassium, and/or carbon; oligo-elements; sea salt; rock salt; clay; steatite; algae; algae extracts; plankton; plankton extracts; plant extracts; roots; liquorice; ginger; oily waxes; proteins; hormones; collagens; alums; alum stone; glucose; vitamins; vitamin C; vitamin A; vitamin F; vitamin E; vitamin B; glycerin; laponite; wetting agents; collagen; acids; salicylic acid; thio acid; caffeine; essential aromatic oils; coloring agents; anti-oxidants; free radical scavengers; moisture absorbers; moisturizers; depigmenting agents; liporegulators; anti-acne agents; antidandruff agents; anti-ageing agents; softeners; antiwrinkle agents; keratolitic agents; anti-inflammatory agents; fresheners; healing agents; vascular protectors; antibacterial agents; antifungal agents; antiperspirants; deodorants; skin conditioners; anesthetics; immunomodulators; and nourishing agents.

In some embodiments, the composite structure 3 may be in a substantially dry form prior to use, for example, thereby rendering it relatively easier to store the composite structure 3. For example, when the composite structure 3 is in such a dry form, the first and second layers 7 and 8 may be characterized by having no significant or relatively little electrical conduction, and the composite structure 3 may be configured to be used when moist.

In the exemplary embodiment schematically depicted in FIG. 1, the electric exciter 2 may include a handle forming box 10, which may serve to house an electricity source, for example, an electric battery 11 (e.g., a 9 volt dry battery as shown schematically in FIG. 8, or a 4.5 volt battery as shown schematically in FIG. 9). Other electricity sources including other types of batteries (e.g., rechargeable batteries) and/or batteries may be used. For example, an adaptor 21 including a transformer as shown in FIG. 10 may be used along with an electric outlet. In some embodiments, voltage delivered by the electrodes 17 and 18 is a direct current (DC) voltage. Alternatively, or in addition, the electric exciter 2 may include, for example, an electronic circuit enabling pulses and/or alternating current (AC) to be delivered.

In the exemplary embodiment depicted in FIG. 1, the box 10 may include a closure cap 12, which may permit the electricity source to be replaced. For example, a thumb tab 13 of the box 10 may render it easier to remove the closure cap 12. The box 10 may include an electric switch 14 and/or a pilot lamp 15, for example, as shown in FIG. 1.

The exemplary electric exciter 2 depicted in FIG. 1 may include a head 16 having two electrodes 17 and 18 projecting from a surface of the head 16. The electrodes 17 and 18 may be held together, for example, via a mass 19 of insulating material, for example, such as a relatively flexible plastic material (e.g., a silicone elastomer). The electrodes 17 and 18 may be electrically connected to the terminals of an electricity source that may be contained in the box 10, for example, via connections not shown.

According to some embodiments, the pilot lamp 15 may be configured to light up when a voltage is applied to the electrodes 17 and 18. Alternatively, or in addition, the pilot lamp 15 may be configured to light up only when an electric current is flowing between the electrodes 17 and 18. In some embodiments, the pilot lamp 15 may be configured, for example, to light up in a first color when the electrodes 17 and 18 are subjected to a voltage and/or may light up in a second color when an electric current is flowing between the two electrodes 17 and 18. For example, the pilot lamp 15 may be a two-color LED.

According to some embodiments, the electric exciter 2 may include electrodes 17 and 18 having various and/or differing shapes. For example, the electric exciter 2 depicted schematically in FIG. 3 includes electrodes 17 and 18 having one or more branches 20. For example, the branches 20 may render it possible to increase the area that is in electrical contact with the composite structure 3 during use.

According to some embodiments, the electrodes 17 and 18 may not have any sharp edges, which may permit them to be displaced (i.e., while in contact with the composite structure 3) without catching on the composite structure 3, and/or which may substantially prevent the electrodes 17 and 18 from scratching the user's skin.

As shown in FIGS. 20 through 25, the electric exciter 2 may include a box 10 housing, for example, a 9 volt electric battery 11. According to some embodiments, the electric exciter 2 shown in FIGS. 20 through 25 may not include an electric switch. According to other embodiments, the electric exciter 2 may include an electric switch. The box 10 may include a closure cap 12 connected to the rest of the box 10 via, for example, a film-hinge 25. Each electrode 17 and 18 may include a rounded head at the end of, for example, a rod 41, and each electrode 17 and 18 may be formed from a conductive elastomer material. The electrodes 17 and 18 may be held (e.g., securely) on the box 10 by a respective set of two walls 20a and 20b, each set defining a mount in which electrodes 17 and 18 may be snap-fastened. The rods 41 of the electrodes 17 and 18 may pass through the top wall of the box 10 and come to bear against the electrical terminals 11a and 11b of the battery 11. A spring or a block of foam 22 may be disposed at the bottom of the box 10, which may permit the battery 11 to be held in electrical contact with the rods 41 of the electrodes 17 and 18.

According to some embodiments, the electrical exciter 2 may be configured in the form of a pen, for example, as shown in FIG. 11. The electrodes 17 and 18 may be concentric 17 and 18 with respect to one another.

According to some embodiments, the electric exciter 2 may include a battery 11 and a head 16. The battery 11 and the head 16 may configured such that the head 16 is mounted on the battery 11, for example, as shown in FIG. 12. In such embodiments, the battery 11 may serve as a handle for manipulating the electric exciter 2.

The electrodes 17 and 18 may be configured to remain stationary relative to the head 16. Alternatively, or in addition, the electrodes 17 and 18 may be adjustably mounted on the electric exciter 2 (e.g., rendering it possible to adjust a distance of separation with respect to one another, for example, as shown in FIGS. 13 through 15, and/or rendering it possible to adjust their orientation with respect to one another, for example, as shown in FIG. 16).

According to some embodiments, for example, as shown in FIGS. 14 and 15, the electrodes 17 and 18 may include rounded ends 22 and may have a generally bowed shape. Some embodiments, such as the one depicted in FIGS. 14 and 15, may include a control member 23, which may render it possible to adjust the voltage at the terminals of the electrodes 17 and/or 18, and/or to adjust the maximum current that may flow from one terminal to the other during use. Some embodiments, for example, the embodiment shown in FIGS. 14 and 15, may include electrodes 17 and 18 configured to pivot about axes (e.g., substantially parallel axes).

According to some embodiments, the electrodes 17 and 18 may be configured to slide. For example, the electrodes 17 and 18 may be engaged in slideways 24, as shown in FIG. 13.

For some embodiments, such as shown in FIG. 17, for example, the electrodes 17 and 18 may be mounted on the box 10 such that (e.g., when not in use) they may be folded and/or rotated into a position that renders it easier to store and/or transport the electric exciter 2.

For embodiments such as the one depicted in FIG. 16, for example, the electrodes 17 and 18 may be independent of one another. For example, the electrodes 17 and 18 may each be fixed on respective electric terminals 11a and 11b of the battery 11. In such embodiments, the battery 11 may secure the electrodes 17 and 18 to one another, for example, so as to permit the electrodes 17 and 18 to be contacted with the composite structure 3 substantially simultaneously. The electrodes 17 and 18 may be electrically connected to contacts 25, which may be configured to be fixed on the terminals 11a and 11b, respectively, of the battery 11.

According to some embodiments, for example, as shown in FIG. 18, the electric exciter 2 may be packaged (and also optionally sold) along with one or more composite structures 3 in a packaging 30, which may be configured, for example, in the form of a case or any other type of package. One or more (e.g., each) composite structures 3 may be packaged individually within the packaging 30 and/or at least some individually-packaged composite structures may be sold separately from the electric exciter 2. According to some embodiments, the electric exciter 2 may be packaged and/or sold individually (i.e., packaged and/or sold independently from the composite structures 3).

During use of the kit 1 according to some embodiments, the user may moisten the composite structure 3, for example, by placing it under either hot or cold running water, and then apply it to the skin on the zone to be treated, for example, as shown in FIG. 4. According to some embodiments, the composite structure 3 may be moistened (e.g., soaked) in a solution other than water, for example, in a salt solution, a solution of water and alcohol, and/or a mixture of water, alcohol, and oil. For some embodiments, adherence of the composite structure 3 to the skin may result solely from the composite structure 3 being moistened (i.e., the composite structure 3 may exhibit no adherence to the skin when dry).

According to some embodiments, once moistened with water, the first and second layers 7 and 8 may permit the reservoir layer 6 to release its active agent(s). The active agent(s) may migrate toward the first surface 4 rather than toward the second surface 5. After initial application of the composite structure 3 to the skin, the electrodes 17 and 18 may not necessarily be in contact with the composite structure 3. Once the composite structure 3 is applied to the skin, the user may bring the electrodes 17 and 18 into contact with the second surface 5 of the composite structure 3 and cause an electric current to pass into the composite structure 3 and into the skin. The passage of electric current into the composite structure 3 and/or into the skin may encourage micro-circulation of the blood and/or it may possibly have an effect on the penetration of the active agent(s) into the skin. Under some circumstances, the user might feel a tingling while the electrodes 17 and 18 are being applied on the composite structure 3, which may result from the current passing into the skin.

The electrodes 17 and 18 may be brought into contact with the composite structure 3 by manipulating the handle 10 of the electric exciter 2. The electric exciter 2 may not need to be displaced relative to the composite structure 3. For example, by not displacing the electric exciter 2 relative to the composite structure 3 treatment of a local area of the skin may be prolonged and/or a very precise zone of the skin may be treated continuously. Alternatively, or in addition, the user may displace the electrodes 17 and 18 over the composite structure 3, for example, so as to vary the locations of the lines of electric current. When the electrodes 17 and 18 are displaced over the skin, displacement may be performed without interrupting the circulation of the current between the electrodes 17 and 18, for example, by keeping the electric exciter 2 in contact continuously against the composite structure 3. The user may also vary the contact pressure of the electrodes 17 and 18 against the composite structure 3, for example, so as to cause more or less electric current to pass through the composite structure 3 and into the skin.

The electric exciter 2 may be applied against the composite structure 3 immediately after the composite structure 3 has been placed on the skin or after a certain amount of time has elapsed. The electric exciter 2 may be withdrawn from the composite structure 3 immediately prior to removal of the composite structure 3 or the composite structure 3 may be left in contact with the skin for a certain amount of time after the electric treatment has ceased.

The composite structure 3 may have at least two methods of use; one without any flow of electric current and another with flow of electric current, which may permit at least two types of treatment to be performed. For example, the user may alternate between treatment that applies electric current and treatment that does not apply electric current. For example, the user may perform a treatment on one day without the contacting the electric exciter 2 to the composite structure 3, and then on the following day, for example, perform a treatment including contacting the electric exciter 2 to the composite structure 3. According to some embodiments of composite structure 3, after treatment, the composite structure 3 may be removed from the skin and discarded.

Displacement of the electric exciter 2 relative to the composite structure 3 may also cause the circulation of the electric current to be interrupted periodically, for example, by moving the electric exciter 2 away from the composite structure 3. For example, the user may successively contact the electric exciter 2 to the composite structure 3. Furthermore, the user may manipulate the switch 14, for example, so as to cause the electric current to pass intermittently into the composite structure 3.

In some embodiments, for example, the exemplary embodiment depicted in FIG. 26, the electric exciter 2 may be configured to transmit vibration to contact members 30 and 31. Contact members 30 and 31 may be configured to operate as electrodes or, alternatively, they may not necessarily be electrically conductive. The exemplary embodiment of electric exciter 28 depicted in FIG. 26 may include electrodes 30 and 31, which can be configured to vibrate via any suitable manner (e.g., via a vibrator 32 included the box 10 that may be at least similar to vibrators found, for example, in mobile telephones, which may include a motor rotating an unbalanced mass). Vibration may be generated in other ways, for example, via an electromagnetic and/or piezoelectric mechanism.

In the embodiment in FIG. 26, the electrodes 30 and 31 are substantially the same shape as the electrodes in the embodiment in FIGS. 20 to 25, but it is not beyond the ambit of the present invention for some other shape to be used.

In the exemplary embodiment shown in FIGS. 27 and 28, an electric exciter 28 may include electrodes 30 and 31 that each include a rod 34 connected to the electric exciter 28. The end of each of the rods 34 may include a substantially spherical head 35 configured contact the composite structure 3. The length of the rods 34 may render it possible to vary the amplitude of the vibrations. For example, the rods 34 may be formed of a spring steel (e.g., a “piano wire”-type spring steel) having a diameter that ranges, for example, from about 0.5 millimeter to about 3 millimeters (e.g., ranging from about 1 millimeter to about 2 millimeters). According to some embodiments, the heads 35 may be formed of a conductive elastomer material, a metal, and/or any other electrically conductive material.

Referring to FIGS. 27 and 28, the electric exciter 28 is schematically depicted in a switched-off condition in FIG. 27 and is schematically depicted in a switched-on condition in FIG. 28. Causing the electrodes 30 and 31 to vibrate may permit the effects of the composite structure 3 on the treated zone to be further increased (e.g., by stimulating the blood circulation locally). For example, as shown in FIGS. 27 and 28, the electric exciter 28 may include a selector 40, which may render it possible for the user to choose between a plurality of differing operating modes (e.g., a mode in which the electrodes 30 and 31 are electrically powered without vibrating, a mode in which the electrodes 30 and 31 vibrate and are electrically powered, and still another mode in which the electrodes 30 and 31 vibrate but are not electrically powered).

According to some embodiments, the electric exciter may include controller (not shown) for adjusting the amplitude and/or the frequency of vibration of the electrodes. The controller may include, for example, a potentiometer and associated electronics in order to vary the voltage and/or frequency of electric power supplied to an electric motor of the vibrator.

Sizes of various structural parts and materials used to make the above-mentioned parts are illustrative and exemplary only, and one of ordinary skill in the art would recognize that these sizes and materials can be changed to produce different effects or desired characteristics.

It will be apparent to those skilled in the art that various modifications and variations can be made to the structure and methodology of the present invention. Thus, it should be understood that the invention is not limited to the examples discussed in the specification. Rather, the present invention is intended to cover modifications and variations.

1. A treatment kit comprising:
an electric exciter comprising at least two electrodes; and
a composite structure not including any electrodes, the composite structure comprising
a first surface configured to be applied to a zone to be treated,
a second surface opposite from the first surface, the second surface being configured to be contacted by the at least two electrodes, and
at least one active agent configured to exert an action on the zone to be treated, the at least one active agent being located between the first surface and the second surface of the composite structure,
wherein the composite structure is configured to encourage migration of the active agent toward the first surface rather than toward the second surface; and
wherein the electric exciter and the composite structure are separate from one another and the treatment kit is configured such that the at least two electrodes can be selectively brought into contact with the composite structure.

2. The kit of claim 1, wherein each of the at least two electrodes is associated with a handle such the at least two electrodes can be brought substantially simultaneously into contact with the second surface of the composite structure via manipulation of the handle.

3. A treatment kit comprising:
a composite structure comprising an outside surface and at least one active agent for exerting an action on a zone to be treated, the composite structure being configured to be made electrically conductive; and
an electric exciter comprising a handle and at least two electrodes secured to the handle such that the at least two electrodes are able to be brought substantially simultaneously into contact with the outside surface of the composite structure, so as to cause an electric current to pass into the composite structure.

4. The kit of claim 3, wherein the composite structure does not include any electrodes.

5. The kit of claim 3, wherein the electric exciter and the composite structure are configured such that the at least two electrodes can come into contact with the outside surface of the composite structure while making no significant contact with the at least one active agent.

6. The kit of claim 3, wherein the composite structure comprises at least one of a patch, a mask, and a cloth.

7. The kit of claim 3, wherein the composite structure comprises a hydrophilic outer layer configured to be contacted by the at least two electrodes.

8. The kit of claim 3, wherein the composite structure comprises at least one layer comprising a woven fabric.

9. The kit of claim 3, wherein the composite structure comprises at least one layer comprising a non-woven fabric.

10. The kit of claim 3, wherein the composite structure comprises two outermost layers, each of the two outermost layers comprising a non-woven fabric.

11. The kit of claim 3, wherein the composite structure comprises at least one layer comprising at least one of a gelled matrix and a polymeric matrix.

12. The kit of claim 3, wherein the composite structure comprises a reservoir layer and two additional layers, the reservoir layer containing the active agent and being located between the two additional layers, at least one of the two additional layers being substantially free from any active agent prior to use.

13. The kit of claim 3, wherein the composite structure comprises a reservoir layer a hydrophilic layer, and a layer that is one of hydrophilic and hydrophobic, the reservoir layer containing the active agent and being located between the hydrophilic layer and the layer that is one of hydrophilic and hydrophobic.

14. The kit of claim 3, wherein the composite structure comprises a reservoir layer and two additional layers, the two additional layers having at least one of differing thicknesses and differing porosities, and the reservoir layer containing the active agent and being located between the two additional layers.

15. The kit of claim 3, wherein the composite structure comprises a reservoir layer, the reservoir layer comprising at least one of the active agent and adhesive.

16. The kit of claim 3, wherein the composite structure comprises a reservoir layer and at least one additional layer, wherein the reservoir layer contains the at least one active agent and the at least one additional layer is bonded to the reservoir layer.

17. The kit of claim 16, wherein the at least one additional layer defines the outside surface of the composite structure, the reservoir layer defines a thickness, and the at least one additional layer defining the outside surface of the composite structure defines a thickness greater than the thickness of the reservoir layer.

18. The kit of claim 16, wherein the at least one additional layer comprises a layer of lower porosity defining the outside surface of the composite structure.

19. The kit of claim 3, wherein the composite structure is substantially dry prior to use.

20. The kit of claim 3, wherein prior to use, the at least one active agent is in at least one of a dehydrated state, a freeze-dried state, and a crystallized state.

21. The kit of claim 3, wherein the composite structure comprises a layer loaded with liquid.

22. The kit of claim 21, wherein the liquid comprises at least one of water, alcohol, and propylene glycol.

23. The kit of claim 21, wherein the layer loaded with liquid comprises the at least one active agent.

24. The kit of claim 3, wherein the composite structure is configured to be non-adhesive when dry and adhesive when dampened.

25. The kit of claim 3, wherein the composite structure comprises magnetic particles.

26. The kit of claim 25, wherein composite structure comprises a layer containing at least a portion of the magnetic particles.

27. The kit of claim 25, wherein the composite structure comprises a reservoir layer containing the at least one active agent and one of a woven fabric comprising fibers and a non-woven fabric comprising fibers, at least a portion of the magnetic particles being at least one of located in the reservoir layer and located in or between the fibers.

28. The kit of claim 3, wherein the composite structure comprises electrically-conductive particles.

29. The kit of claim 28, wherein the composite structure comprises metallic particles.

30. The kit of claim 28, wherein the composite structure comprises copper particles.

31. The kit of claim 3, wherein the active agent comprises at least one compound selected from metals; metal alloys; cobalt; barium; chromium; aluminum; silver; copper; titanium; bronze; manganese; metallic oxides; iron oxides; ferrites; rare earths; silicates; sulfates, barium sulfates; carbonates; calcium carbonates; non-ferrous compounds; sulfur; magnesium; calcium; boron; potassium; carbon; oligo-elements; sea salt; rock salt; clay; steatite; algae and plankton and extracts therefrom; roots; liquorice; ginger; oily waxes; proteins; hormones; collagens; alums; alum stone; glucose; vitamins; vitamin C; vitamin A; Vitamin F; vitamin B; vitamin E; plant extracts; glycerin; laponite; wetting agents; collagen; acids; salicylic acid; thio acid; caffeine; essential aromatic oils; coloring agents; anti-oxidants; free radical scavengers; moisture absorbers; moisturizers; depigmenting agents; liporegulators; anti-acne agents; antidandruff agents; anti-ageing agents; softeners; antiwrinkle agents; keratolitic agents; anti-inflammatory agents; fresheners; healing agents; vascular protectors; antibacterial agents; antifungal agents; antiperspirants; deodorants; skin conditioners; anesthetics; immunomodulators; and nourishing agents.

32. The kit of claim 3, wherein the composite structure comprises a reservoir layer containing the at least one active agent, the reservoir layer comprising at least one of a super-absorbent compound; a polyacrylate; an acrylic polymer; a vinyl polymer; a polyurethane; a latex; and a pseudolatex.

33. The kit of claim 32, wherein the composite structure comprises glycerin.

34. The kit of claim 3, wherein the at least two electrodes are associated with the electric exciter such that the at least two electrodes may be displaced relative to the electric exciter.

35. The kit of claim 34, wherein the at least two electrodes are configured to be displaced relative to the electric exciter via at least one of sliding and turning.

36. The kit of claim 3, wherein the at least two electrodes are separated by a distance from one another, the distance being adjustable.

37. The kit of claim 3, wherein at least one of the electrodes is configured to be removed from the electric exciter.

38. The kit of claim 3, wherein the at least two electrodes are configured to be prevented from moving relative to one another other via an electrical insulator.

39. The kit of claim 38, wherein the electrical insulator comprises a non-conductive material selected from elastomer, thermoplastic resin, and silicone.

40. The kit of claim 3, wherein the at least two electrodes are substantially free of sharp edges.

41. The kit of claim 3, wherein each of the at least two electrodes comprises a rounded end.

42. The kit of claim 3, wherein the electric exciter defines an ergonomic shape.

43. The kit of claim 3, wherein the handle at least partially comprises a battery.

44. The kit of claim 3, wherein the electric exciter comprises a box configured to house at least one battery.

45. The kit of claim 3, wherein the electric exciter is configured to be connected to an electrical adaptor connected to an electric outlet.

46. The kit of claim 3, wherein the at least two electrodes each comprise a terminal and the electric exciter is configured such that voltage at the terminals of the at least two electrodes is substantially identical to voltage at terminals of an electricity source associated with the electric exciter during use.

47. The kit of claim 3, wherein the electric exciter is configured such that voltage at terminals of the at least two electrodes is less than about 30 volts.

48. The kit of claim 3, wherein the electric exciter comprises a control member configured to adjust electrical power.

49. The kit of claim 3, wherein the composite structure comprises a patch through which electric current can flow to the skin.

50. The kit of claim 3, wherein the electric exciter comprises a vibrator configured to cause the at least two electrodes to vibrate.

51. The kit of claim 50, wherein the electric exciter comprises a selector configured to allow a user to selectively activate the vibrator.

52. The kit of claim 50, wherein the electric exciter comprises a selector configured to allow a user to choose an operating mode in which the at least two electrodes vibrate without being electrically charged.

53. An electric exciter comprising:
a battery;
at least two electrodes; and
a head configured to be rigidly secured to the battery,
wherein the head is associated with the at least two electrodes such that the at least two electrodes are prevented from moving relative to one another, and the head being configured to allow the at least two electrodes to be brought into substantially simultaneous contact with a composite structure.

54. An electric exciter comprising:
a battery;
two electrodes;
a vibrator configured to cause the two electrodes to vibrate; and
a selector configured to selectively control the vibrator.

55. The electric exciter of claim 54, wherein the two electrodes comprise two conductors each provided with a rounded head.

56. The electric exciter of claim 55, wherein the two conductors each comprise thin rods.

57. An electric exciter comprising:
a box comprising a plastic material and being configured to receive at least one battery having terminals; and
two electrodes associated with the box, each electrode comprising an external rounded head and a rod configured to contact a respective terminal of the battery.

58. A cosmetic treatment method for cosmetically treating a zone of the skin, the method comprising:
applying a composite structure to the zone of the skin to be treated, the composite structure comprising at least one active agent and including no electrodes; and
contacting the composite structure with an electric exciter comprising at least two electrodes, the composite structure being configured to pass electric current into the composite structure without significant contact occurring between the at least two electrodes and the at least one active agent.

59. The method of claim 58, further comprising dampening the composite structure before applying the composite structure to the zone to be treated.

60. The method of claim 58, wherein the composite structure is configured to become adhesive upon contact with the skin.

61. The method of Claim 60, further comprising dampening the skin prior to applying the composite structure to the zone of the skin to be treated.

62. The method of claim 61, further comprising dampening the skin before applying the composite structure in a dry state to the zone of the skin to be treated.

63. The method of claim 58, wherein during treatment, the at least two electrodes are prevented from moving relative to the composite structure.

64. The method of claim 58, wherein during treatment, the at least two electrodes are moved relative to the composite structure.

65. The method of claim 58, wherein the at least two electrodes are in contact with the composite structure continuously during treatment.

66. The method of claim 58, wherein the at least two electrodes are in contact with the composite structure intermittently during treatment.

67. The method of claim 58, wherein the at least two electrodes are in contact with the composite structure for a period of time ranging from about 3 minutes to about 15 minutes.

68. The method of claim 58, wherein the composite structure is configured to be discarded after use.

69. The method of claim 58, wherein during treatment, the at least two electrodes are caused to vibrate.

70. A cosmetic treatment method for cosmetically treating a zone of the skin, the method comprising:
dampening a composite structure that does not have electrodes and that comprises at least one active agent; and
bringing at least one electrode into contact with the composite structure so as to cause an electric current to pass through the composite structure.

71. The kit of claim 1, further comprising a packaging, wherein the electric exciter and the composite structure are packaged together within the packaging.
